(19) United States
(12) Patent Application Publication
(10) Pub. No.: US 2021/0398633 A1
(43) Pub. Date: Dec. 23, 2021

(54) SYSTEMS AND METHODS FOR TRANSACTING PRESCRIPTIONS USING A MOBILE DEVICE

(71) Applicant: Harrow IP, LLC, Nashville, TN (US)

(72) Inventors: Mark L. BAUM, Nashville, TN (US); Andrew E. Livingston, Nashville, TN (US); Feliks Dushatsky, Nashville, TN (US)

(21) Appl. No.: 17/355,018

(22) Filed: Jun. 22, 2021

Related U.S. Application Data
(60) Provisional application No. 63/042,455, filed on Jun. 22, 2020.

(51) Int. Cl. G16H 20/00; G16H 10/60; G16H 40/67; G06K 9/00

(52) U.S. Cl. CPC G16H 20/00; G16H 10/60; G06K 2209/01; G06K 9/00442; G16H 40/67

(57) ABSTRACT

Some embodiments of the present disclosure disclose methods and systems for issuing and filling medical prescriptions for a patient. In some embodiments, the system may include a mobile device for use by the medical prescriber in cellular communication with a computer network that in turn is coupled to a computer server. The computer server may include an authentication engine configured to authenticate the identity of a prescriber of a medication, medical device, medical treatment, etc., and a prescription engine configured to generate a prescription based on a message from the mobile device that may be in the form of an image, audio file, text message, etc., based on the result of the authentication.

### CROSS-REFERENCE TO RELATED APPLICATIONS

The present application claims priority to and the benefit of the U.S. Provisional

Patent Application No. 63/042,455, filed Jun. 22, 2020, titled “Systems and Methods for Transacting Prescriptions Using a Mobile Device,” which is hereby incorporated by reference in its entirety as if fully set forth below and for all applicable purposes.

### TECHNICAL FIELD

The present application generally relates to systems and methods for issuing and filling medical prescriptions for a patient, and more particularly, to improved systems and methods for issuing and filling medical prescriptions using one or more mobile devices.

### BACKGROUND

Currently it's not a simple process or user experience for doctors to send customized prescriptions to a pharmacy. Currently, some doctors use their electronic medical records (EMR) software and/or e-prescribing applications like that available under the trademark SURESCRIPTS® from Surescripts, LLC of Arlington, Va., to submit a prescription to a pharmacy. However, these e-prescribing tools are relatively complex, and often require outside help to support them.

In addition, it is sometimes difficult for prescribing physicians, when attempting to use current e-prescribing tools, to find certain medications, especially when such medications are customized to the patient and require separate compounding by a compounding pharmacy. Compounding pharmacies fall outside of the traditional pharmacy workflow for most EMR software and e-prescribing tools. In the case of specially-compounded medications or personalized medications, pharmacies often require 100% front-end payment by the patient, compared with a co-pay backed by medical insurance plan benefits.

Moreover, in those cases wherein a health care provider must use both EMR software and a separate electronic-prescription software tool to issue a prescription, information regarding the patient and the prescription being prescribed may need to be separately entered into both such systems, requiring extra time.

Current electronic prescribing tools are designed to be operated via an office desktop computer with Internet access. If the prescriber is present in the prescriber's office, then such tools may be used. However, if the prescriber is away from the office, the prescriber may not be able to issue a prescription. In some cases, the prescriber can authorize another doctor, a physician's assistant, nurse or medical assistant who is present in the office to issue a prescription on behalf of the prescriber. However, in the case of controlled substances, this may not be permitted, resulting in the delay of the issuance of the prescription.

Any system used by doctors to issue prescriptions must comply with federal HIPAA laws to protect the privacy of patients. In addition, in the case of controlled substances, pharmacies must be reasonably assured that the prescription is genuine, and that the prescriber was authorized to issue such prescription.

Many patients want to take an active role in their health care and wish to be advised, in real time, as to the status of the issuance of, and filling of, their prescriptions. Currently, however, patients may be unaware that a prescription has been issued until they are contacted by their pharmacy.

Preparation of prescriptions by a pharmacy are often delayed until the patient contacts the pharmacy to arrange for payment and pickup or delivery. Delays in getting the prescribed medication into the patient's possession can compromise the patient's health.

### SUMMARY OF THE INVENTION

It is therefore an object of the present invention to provide a system and method for allowing physicians and/or their authorized assistants to efficiently issue prescriptions to a pharmacy for their patients, wherein such system and method are relatively simple to use.

Another object of the present invention is to provide such a system and method which keeps patients regularly updated relative to the issuance of such prescriptions, and when the prescribed medication is accessible by the patient.

Still another object of the present invention is to provide such a system and method for allowing prescribing doctors to create and issue prescriptions even when they are away from their office.

Yet another object of the present invention is to simplify the process for a prescriber to specify particular medications to be compounded for a patient.

It is also an object of the present invention to comply with applicable HIPAA laws to protect the privacy of patients, and to ensure that prescriptions issued for controlled substances are genuine and issued by an authorized person.

A further object of the present invention is to provide to promptly notify patients when a prescription has been issued.

A still further object of the present invention is to expedite preparation of the prescribed medication by a pharmacy and avoid delays resulting from the need for the pharmacy and the patient to arrange terms for payment.

These and other objects of the present invention will become more apparent to those skilled in the art from the description below.

Briefly described, the disclosed embodiments relate to a system and method for allowing a prescriber, who may be a doctor or other health care provider, to issue a prescription on behalf of a patient to a pharmacy for medication, to a medical equipment supplier for a medical device, or to another health care provider for medical treatment, using a mobile device, such as a smartphone or cellular-equipped tablet.

In accordance with various embodiments, a prescription system allows a doctor or other medical prescriber to issue a medical prescription to a supplier of medication, medical devices, or medical treatment, using the prescriber's cellular smartphone or other cellular-compatible mobile device. The prescriber's mobile device includes a cellular transceiver for communicating with a cellular communications network. The prescriber's mobile device is adapted to transmit and receive electronic messages (e.g., SMS or MMS messages) over the cellular communications network. Such electronic messages transmit information identifying the prescriber, the patient, and an item being prescribed, e.g., a medication, a medical device, or further medical treatment, for the benefit of the identified patient. A computer network couples the cellular communications network to a computer server having a data processor and data storage. The computer server communicates, via the computer network and cellular communications network, with the prescriber's mobile device. In various embodiments, the computer server also communicates with an electronic device of the identified patient.

The aforementioned computer server further includes authentication logic, e.g. an authentication engine, configured to authenticate the identity of the prescriber. The computer server further includes prescription logic for generating a prescription for the identified patient in accordance with information transmitted by the prescriber's mobile device. The computer server transmits the generated prescription over a computer network to a supplier of the prescribed medication, prescribed medical device, or prescribed medical treatment. In various embodiments, the prescription logic identifies a supplier of the prescribed item that is in closest proximity to the identified patient. In other embodiments, the prescription logic may identify the supplier that is best qualified to make a particular prescribed medication or item. In still other embodiments, the prescription logic may identify the supplier who offers the lowest price for the prescribed medication or item. In various embodiments, the computer server also provides payment information for the identified patient and transmits the generated prescription and payment information over a computer network to the supplier.

In other embodiments, a method allows a prescriber to issue a medical prescription on behalf of a patient to a supplier of an item of medication, medical devices, or medical treatment. In practicing such method, a prescriber uses a mobile device in his or her possession to transmit information identifying the prescriber over a cellular communications network. This information identifying the prescriber is transmitted from the cellular communications network to a computer server. The computer server compares the prescriber identification information transmitted by the prescriber to authentication information stored in the computer server to authenticate the identity of the prescriber. Using his or her mobile device, If the prescriber is properly authenticated, the medical prescriber transmits information identifying a patient and an item being prescribed for such patient over the cellular communications network. The information identifying the patient and the item being prescribed for such patient is transmitted from the cellular communications network to the computer server. A prescription is generated in electronic format for the identified patient by the computer server. The generated prescription in electronic format is then transmitted from the computer server to a supplier of the item being prescribed. In various embodiments, the computer server identifies a supplier of the prescribed item that is in closest proximity to the identified patient, that is best qualified to fill the prescription, that has the prescribed item in stock, or that offers the prescription at the lowest price. In various embodiments, the computer server provides payment information for the identified patient, and such payment information is transmitted to the supplier along with the generated prescription.

The foregoing and other features and advantages of the present invention will become more apparent from the following more detailed description of particular embodiments of the invention, as illustrated in the accompanying drawings.

### BRIEF DESCRIPTION OF THE DRAWINGS

A more complete understanding of the present invention may be derived by referring to the detailed description and claims when considered in connection with the Figures, wherein:

FIG. 1 is a simplified block diagram illustrating a system for issuing and filling medical prescriptions using mobile devices according to various aspects of the present disclosure.

FIG. 2 is a simplified flowchart illustrating the steps performed by the system of FIG. 1 to allow a prescriber to issue a prescription for the prescriber's patient according to various aspects of the present disclosure.

FIG. 3 illustrates an example neural network that can be used to implement a computer-based model according to various embodiments of the present disclosure.

FIG. 4 is a flowchart illustrating a method for issuing a prescription for a prescriber's patient according to various aspects of the present disclosure.

FIG. 5 is an example computer system, according to various aspects of the present disclosure.

### DETAILED DESCRIPTION

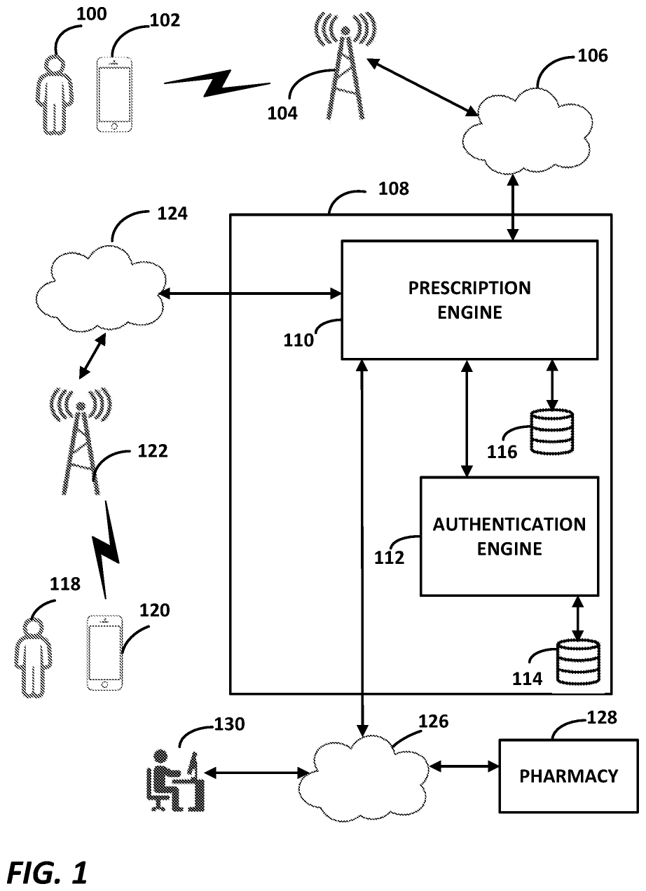

Referring to FIG. 1, prescribing doctor 100 operates mobile device 102. Mobile device 102 may be a smartphone that is capable of communicating with local cell tower 104 for exchanging data over network 106. Mobile device 102 may also be a tablet equipped for cellular communications or any other mobile device so equipped. Mobile device 102 includes a cellular transceiver for communicating with a cellular communications network to which local cell tower 104 is linked. Mobile device 102 is adapted to transmit and receive electronic messages, including SMS text messages and/or MMS multimedia messages.

Network 106 is coupled to automated prescription system 108. System 108 may be provided in the form of a backend, cloud-based network server of the type that includes a data processor and associated data storage. System 108 includes a prescription engine 110 (also described herein as prescription logic) for receiving prescriptions issued by doctor 100, as well as other subscribing doctors, to verify and create prescriptions. As shown in FIG. 1, system 108 also includes an authentication engine 112 (also described herein as authentication logic) which is configured to authenticate the identity of a doctor or other person requesting issuance of a prescription to a pharmacy. A data storage 114 is coupled to authentication engine 112 for storing data used to authenticate prescribers requesting issuance of a prescription. For example, data storage 114 may be used to store passwords or PIN codes uniquely assigned to prescribers making use of the system.

Data storage 114 may also be used to store government-issued controlled substance prescribing numbers assigned to each prescriber. Data storage 114 may also be used to store valid telephone numbers for each prescriber. Data storage 114 may also be used to store prescriber's facial image, eye iris image, fingerprints, thumbprints, voice sample, and/or other biometric information that may be used to verify the authenticity of the prescribing physician. In some cases, geo-location data may be stored in data storage 114 corresponding to the location(s) of the doctor's office(s), or the doctor's residence.

In at least one embodiment, prescribing doctor 100 would first download a software app, sourced by system 108, onto the user's mobile device 102 before using automated prescription system 108 for the first time. Once downloaded onto mobile device 102, this software app may permit doctor 100 to select and use a customized keyboard when texting information to the automated prescription system. The software app installed on the user's mobile device 102 can create a text shortcut that doctor 100 can press to open a menu of options.

Apart from entering data into the software app installed on the doctor's mobile device 102 by pressing characters on a displayed keyboard, doctor 100 could also record an audio file on the user's mobile device, wherein the audio message includes all of the information needed to create a legal medication prescription. For example, the recorded audio file could include the doctor's name, password or PIN code, the name and date of birth of the patient, the medication and dosage being prescribed, and identification of the pharmacy.

In yet another embodiment, a prescriber can write out a prescription from a prescription pad, take a picture of such hardcopy prescription using the built-in camera of his or her mobile device 102, and then text the picture of such hardcopy prescription (e.g., as a .jpg file) to the system 108 as an attachment to such text, similar to the manner in which doctors currently fax hardcopy prescriptions to a pharmacy. If desired, the image of the prescription being transmitted by text message could already be in Portable Document Format (.pdf).

In still another embodiment, the details of the prescription, including doctor name, password or PIN code, patient name, date of birth, medication and dosage, and pharmacy, could all be transmitted to system 108 as conventional text messages. Even if such texted information were not in a specified format, the backend server may include an artificial intelligence engine to interpret the information transmitted by the prescriber, and text the same information in a standardized format back to the prescriber for review and confirmation by the prescriber.

For example, in the case of a texted audio file, system 108 would apply such artificial intelligence to transcribe the audio message into written words and determine which words belong in which fields (doctor's name, patient's name and date of birth, etc.). The formatted data would be texted back to the doctor's mobile device 102 for review by doctor 100, and a request for the doctor to text back a confirmation that the formatted data is correct.

Likewise, if doctor 108 texts a graphic image (e.g., .jpg, .pdf, etc.) of a prescription by to system 108, then prescription engine 110 applies optical character recognition (OCR) software to convert the transmitted image into text. The artificial intelligence engine module of prescription engine 110 would allocate such text into standardized formatted fields for texting back to doctor 110 for confirmation of accuracy. In the case of a prescription transmitted using standard text messaging, the text is already available, and the artificial intelligence engine would simply allocate such textual information into the appropriate formatted fields, and text the result back to doctor 100 for confirmation.

In regard to the use of artificial intelligence to process data submitted by the prescriber, a prescriber data intake engine, or module, may be configured to receive the prescriber's submitted data as unstructured data, convert the unstructured data into structured data according to a predefined form for arranging prescriber data and making predictions based on an analysis of the prescriber data. In some embodiments, the term “unstructured data” refers to data that may not be organized in a pre-defined pattern or manner, such as but not limited to free-flowing speech or voice data, text that is not arranged in a standard format, documents such as photo images of hardcopy prescription forms, videos, etc.

Artificial intelligence, implemented with neural networks and deep learning models, has demonstrated great promise as a technique for automatically analyzing real-world information with human-like accuracy. In general, such neural network and deep learning models receive input information and make predictions based on the same. Whereas other approaches to analyzing real-world information may involve hard-coded processes, statistical analysis, and/or the like, neural networks learn to make predictions gradually, by a process of trial and error, using a machine learning process. A given neural network model may be trained using a large number of training examples, proceeding iteratively until the neural network model begins to consistently make similar inferences from the training examples that a human might make. Neural network models have been shown to outperform and/or have the potential to outperform other computing techniques in a number of applications.

In some embodiments, the prescription engine 110 may be configured to include a chatbot for receiving audio data as input data. Thus, a doctor may be able to speak into mobile device 102 and prescription engine 110 may process the unstructured voice data that includes doctor authentication data, patient identifying data, data identifying the medication or item being prescribed, and data relating to selection of an appropriate pharmacy or supplier, for converting the (unstructured) voice data into data that is structured according to a desired standardized format. The AI intake module receives and processes the voice data to convert the voice data into structured data in a standardized format. In some cases, the prescriber may state information without being prompted by the chatbot, while in other cases, the chatbot may pose the questions to prompt the prescriber to provide the answers. In some embodiments, the audio input data may be in the form of a recorded voice data (e.g., voice recording, video, etc.). In such cases, the AI intake module may extract the voice data from the recording and process the voice data to convert the voice data into a structured data as discussed above.

In some embodiments, the intake module may be in the form of a chatbot that is configured to receive visual data as input data. In some cases, the visual data can be text data, image data, video data (i.e., a series of image data), etc., and the intake module may be configured to receive and/or extract such visual data. For example, the prescriber may present a photograph of a hardcopy prescription to the intake module, which may be configured to extract from the photographic image at least a substantial portion of the text, image, etc., as input data (e.g., scan the documents and identify the relevant text, image, etc.). As another example, the visual data can be a video including voice and image data related to the prescriber, the patient, the prescribed medication and dosage, and even the preferred supplier. The intake module may then extract such information to be processed using artificial intelligence for conversion into structured data as discussed above.

In some embodiments, the AI intake module may include or be coupled to additional engines. For example, one of such additional engines may include a natural language processing (NLP) engine configured to analyze or parse natural language data such as text, images, etc., of the input data and convert such (unstructured) data into structured data. Another example of such additional engines includes a video parsing engine configured to analyze (e.g., segment, index, etc.)

the video input data to identify information from the video and convert the same into a structured form, e.g., populate a structured form with information extracted from the video input data as a result of the analyses by the video parsing engine. In some cases, the AI intake module may include or be coupled to a voice recognition engine configured to extract the voice of the prescriber from the input data and identify the identity of the prescriber (e.g., based on a comparison of the extracted voice to voice samples stored in a prescriber records database in communication with the intake module). Other examples of engines that are part of, or coupled to, the AI intake module include ANI (Artificial Narrow Intelligence), ASI (Artificial Super Intelligence), DL (Deep Learning), ML (Machine Learning), and NLP (Natural Language Processing).

In some embodiments, any of the above noted engines may be implemented by an automated software. For example, the authentication engine may have a software or module therein that is configured to receive information or data about a prescriber to automatically compare the received information with an authentication information of the prescriber that is stored in the database 114 to authenticate the identity of the prescriber. In some instances, the prescription engine may have a software or module therein that receives information (e.g., a text message) about a patient and item that is being prescribed to the patient, and generate a prescription for the item by populating fields of a form prescription. In some embodiments, the authentication engine and/or the prescription engine may be artificial intelligence (AI) powered. For example, the prescription engine can be a neural network engine that is trained to receive image files, audio files, video files, text messages, etc., transmitted by mobile devices of prescribers and extract from the received files/messages prescriber, patient, prescribed item, etc., so as to populate the extracted information into the fields of a prescription form to generate a prescription in an electronic format. In some instances, the prescription engine can include or be coupled to other AI -powered engines which may be used in the extraction of the above-noted information. For example, the prescription engine may include or be coupled to an OCR engine and/or a NLP engine that is configured to extract text from image files. In some instances, the prescription engine may include or be coupled to a voice recognition engine that is configured to extract text from voice or audio files. That is, if a prescriber transmits a file (e.g., an image file, voice/audio file, video file, text message, etc.,) including or narrating identifying information about the prescriber itself (e.g., the prescriber's name, identification number, etc.), the patient (e.g., name, age, patient identification no.), item to be prescribed (e.g., types, amount, etc.), the prescription engine may utilize any of the other AI-powered engines (e.g., OCR engine, NLP engine, etc.) to extract the identifying information from the file/message so as to populate the fields of a prescription form with the identifying information to generate a prescription in an electronic format.

As already noted, security is an important issue with such a prescription system, particularly when complying with Federal HIPAA laws and regulations. If a prescriber is using a conventional smartphone to issue the prescription, then use of system 108 may be conditioned upon the doctor's smartphone being password protected and/or require local facial recognition of the doctor's face before mobile device 102 may be used. System 108 may require a user to enter a four digit PIN code, uniquely assigned to the user's mobile device 102, before doctor 100 is allowed to make use of system 108. System 108 may be configured to require a prescriber to enter his or her government licensed controlled substance prescribing number before authorizing issuance of a prescription, particularly when doctor 100 is attempting to issue a prescription for a controlled substance. The system may also check whether the mobile device being used to place the prescription has an assigned telephone number that matches with the mobile device being used by the prescriber who is placing the prescription. As an added level of security, a user's face, eye iris, fingerprint, thumbprint, voice or other biometric information may be used to verify the authenticity of doctor 100. For example, doctor 100 may be required to use the camera of his or her mobile device to create an image of the user's face, and to text such image to system 108 for comparison with previously stored images stored in data storage 114 to confirm the authenticity of the user. The user's geo-location may also be used for security purposes, wherein the prescriber would need to be communicating through a cell tower located proximate to the prescriber's designated office(s) or proximate to the prescriber's home. These extra security measures could be reserved for highly-controlled medications, e.g., those containing narcotics.

While it is possible that some or all of the security authentication procedures described above could be performed on the user's mobile device 102, through an installed software “app”, it may be advantageous for such security authentication procedures to be carried out by software installed in authentication engine 112 (i.e., at the enterprise software level), since this would be more difficult to circumvent and would avoid the need to complicate and burden the software app installed on the user's mobile device 102. As already noted, data storage 114 may be used to store a variety of authentication information, including voice samples, facial images, passwords, PIN codes, other biometric information, etc. Authentication engine 112 can then compare such stored information with authentication information texted by doctor 100 to determine whether doctor 100 is the person actually operating mobile device 102. Authentication engine 112 then notifies prescription engine 110 that the identity of doctor 100 has been verified, and that prescription engine 100 may proceed with the processing of such prescription.

In those instances where one or members of the physician's staff are authorized to issue prescriptions for certain medications on behalf of a physician, the same verification techniques may be used to verify the identity of the person requesting issuance of such prescription to the pharmacy.

Each prescribing doctor can maintain a “favorites” list of drugs typically prescribed for the types of patients usually seen by such physician. For example, an ophthalmologist or eye surgeon might save a preferred list of medications typically prescribed for patient's eyes. This preferred list of medications can be saved, for example, in data storage device 116 for display on the screen of mobile device 102. Alternatively, a group of graphical images of labels of drugs commonly prescribed by each such physician may be saved in data storage 116 for display on the user's screen for allowing the prescriber to quickly select a desired medication and dosage. In addition, data storage 116 may save a wide variety of medications in standardized dosages from which the prescriber may select.

To assist doctor 100 in specifying a desired pharmacy, doctor 100 can text the patient's zip code to system 108, and system 108 can text back to doctor 100 choices of pharmacies located near the patient; a list of available pharmacies indexed by zip code may be saved in data storage 116. The doctor may then text an identifying pharmacy ID number back to system 108 corresponding to the preferred pharmacy. This option would not be required if prescription engine 110 has previously been advised of a patient's preferred pharmacy. Such patient preferences may be stored, for example, in a data storage device 116 coupled to prescription engine 110.

To ensure that doctor 100 has in mind the correct patient when issuing a prescription, system 108 can transmit by text to mobile device 102 an image of the patent's driver's license, or other form of patient identification, for display on the display screen of mobile device 102, and the system may then ask doctor 100 to confirm that the currently selected patient is correct. Optionally, prescription engine 110 may also save information regarding each patient's allergies and past prescriptions, which information may be stored in data storage 116. This information could be displayed to doctor 100 on mobile device 102 when displaying patient identification information for confirmation.

Still referring to FIG. 1, when prescription engine 110 has performed all required authentications and doctor 100 has confirmed via text that the prescription created by system 108 is correct, prescription engine 110 sends a confirmation of the issuance of the prescription over network 106 to doctor 100. At the same time, prescription engine 110 sends a text message over network 124 to a cell tower 122 in communication with mobile device 120 of patient 118. In this way, patient 118 is informed in real time that such prescription has been issued. In addition, confirmation of the issuance of such prescription is sent over network 126 to the pharmacy 128 specified by doctor 100 (or the pharmacy previously stored as the patient's preferred pharmacy).

To ensure that prescribers have adequate technical support, and to detect and resolve any unexpected problems, human tech support (130 in FIG. 1) may be connected to system 108 over network 126 to monitor every prescription being generated. This may include actual observation of every conversation between doctor 100 and system 108, and the ability for human tech support 130 to send messages to, and receive messages from, doctor 100 for quickly responding to any inquiry or request for support. Human tech support advocate 130 can be notified when certain conditions exist, as when a prescriber is having difficulty providing correct patient identification information, when a prescriber repeatedly fails to successfully submit authenticating credentials, or when a prescriber attempts to select a pharmacy or other supplier that does not supply the medication or other item being prescribed. When appropriate, human tech support advocate 130 could contact further support staff when needed.

To facilitate payment of pharmacy 128 for preparing and filling the prescription, a copy of the patient's credit card may be stored within system 108, for example, within data storage 116. This information may be provided to pharmacy 128 at the same time that the prescription is transmitted to pharmacy 128 to avoid any delays in processing the prescription. Alternatively, when prescription engine 110 transmits the prescription to pharmacy 128, a text message may be sent to patient 118 to notify the patient that a prescription has been issued to the selected pharmacy, displaying the stored credit card information and the anticipated charges for such medication, and asking the patient to confirm, via text message, the patient's authorization to use such credit card to pay the anticipated pharmacy charges.

In those instances where a doctor/prescriber is pre-occupied with another activity that prevents the prescriber from operating buttons on his or her mobile device 102, e.g., when the prescriber is driving a vehicle, the issuance of the prescription may be effected by a conventional telephone call to a voice-responsive portal of system 108. In this case, the doctor can be prompted to verbally respond to commands requesting each piece of necessary information, and system 108 can create an audio file, for archiving such telephone call in data storage 116, in the event that an audit is later conducted, or a question otherwise arises as to whether such prescription was properly issued. If desired, the system can compare such audio file to previously-stored audio samples by such prescriber to confirm that an authorized prescriber is actually issuing the current prescription. Alternately, the prescriber's mobile device may be used to establish a video conference with the pharmacy, and the video images of the prescriber may be archived for later reference.

When pharmacy 128 has prepared the prescription and it is ready for the patient, pharmacy 128 can send a message over network 126 to system 108, which then sends a message to patient 118, and optionally to doctor 100 as well, advising that the prescription is ready, and confirming payment details. The patient may then pick-up the prescription from the pharmacy or the medication can be shipped by the pharmacy to the patient. System 108 then updates the records maintained by the pharmacy for such patient to record the issuance and filling of such prescription. Simultaneously, system 108 provides an update for the medical records maintained by the prescriber for such patient regarding the issuance and filling of such prescription. This update may be transmitted via a hypertext link, a telefax transmission, a text message transmission, an audio .wav file, or an email message to the doctor's office. The management software in the prescribing physician's office responds to such update by recording the issuance and filling of such prescription for such patient. If desired, system 108 may be used to maintain electronic medical records on behalf of both the prescribing doctor and the pharmacy relative to the particular patient in issue.

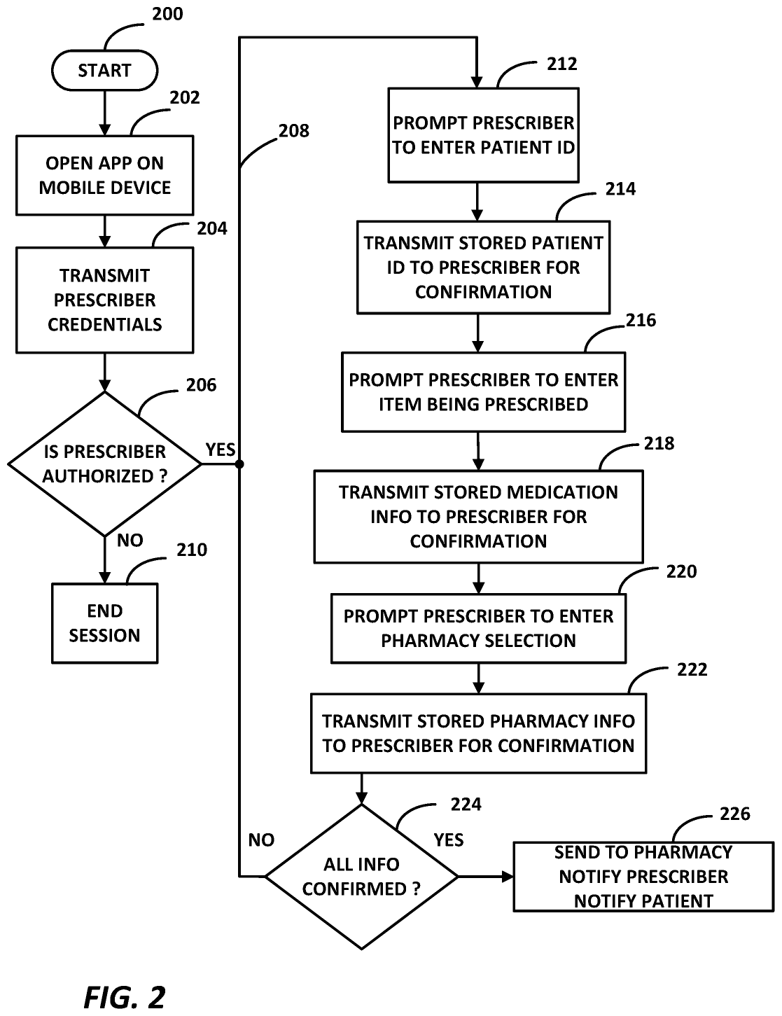

Now referring to FIG. 2, a simplified flowchart is set forth which illustrates the basic method steps performed by the system embodiment shown in FIG. 1. Control begins at Start block 200. Before a prescriber proceeds to request issuance of a prescription, the prescriber first opens an app (application software) on his or her mobile device 102, as represented by block 202. The app then prompts the prescriber to enter his or her authentication credentials (PIN code, facial image, thumbprint, etc.), after which the app transmits such authentication credentials to system 108, as represented by block 204. Upon receiving the transmitted authentication credentials from the prescriber, authentication engine 112 of system 108 compares such credentials to authentication information stored in data storage 114, as represented by decision diamond 206. If there is a match, then the prescriber is authorized, and control passes along line 208 to block 212. If there is not a match, then the session is ended, as represented by block 210.

Still referring to FIG. 2, following successful authentication, prescription engine 110 of system 108 sends a message back to mobile device 102 prompting doctor 100 to enter information identifying the patient for whom such prescription is to be generated, as represented by block 212. In response, doctor 110 enters patient identification information into mobile device 102 and transmits it back to prescription engine 110. Upon receipt of such patient identification information, prescription engine may run a comparison of the received information to a stored database of patients registered to the prescribing doctor. Assuming that such stored patient information is found, the stored information (which may include an image of the patient's driver's license) is transmitted by system 108 back to mobile device 102 for confirmation by doctor 100, as indicated by block 214. After doctor 100 confirms the patient information, mobile device 102 prompts doctor 100 to enter the medication being prescribed, as reflected by block 216. This may include a button labeled “Favorites” to recall a menu of saved medications that doctor 100 commonly prescribes. Otherwise, doctor 100 may enter the name of a medication, and a search can be made of medications stored in data storage 116 of prescription engine 110 for display on mobile device 102. After doctor 100 selects a desired medication, prescription engine 110 may send a request back to mobile device 102 asking doctor 100 to confirm the medication/dosage to be prescribed, as represented by block 218. Mobile device 102 then prompts doctor 100 to select a pharmacy at which the prescription is to be filled, as indicated by block 220. In this instance, doctor 100 may be presented with a series of choices suggested by prescription engine 110 based upon the patient's zip-code or by a previously-stored patient preference. In the case of specially-compounded medications, the doctor may have stored “Favorite” compounding pharmacies from which the doctor may select. After doctor 100 makes selection, prescription engine 110 sends a message back to mobile device 102 requesting the doctor to confirm the pharmacy selection, as represented by block 222. As already noted, selection of a desired pharmacy or supplier may be based upon the proximity to the patient. Alternatively, prescription engine 110 may suggest a pharmacy or supplier that is best qualified to make a particular prescribed medication or item; for example, a prescription for a medication that will require greater skill and expertise to compound properly would be best directed to a compounding pharmacy that has significant experience in compounding medications of such type. This information could be stored in data storage 116 for various medications and pharmacies, and such information could be displayed to the prescriber before the prescription is issued. Another option is for the prescription logic to suggest a supplier who offers the lowest price for the prescribed medication or item; this information could also be stored in data storage 116 and updated regularly. If desired, prescription engine 110 could rank available pharmacies based upon both proximity to the patient and price, with the understanding that most patients would prefer to drive an extra mile or two to pick-up a prescription if they save money by doing so. In addition, data storage 116 could be used to store information regarding whether particular medications or items are currently in stock at particular pharmacies or suppliers, and cause a warning to be displayed to a prescriber if a pharmacy or supplier tentatively selected by the prescriber is out of stock on that medication or item; data storage 116 would be continuously updated by participating pharmacies and suppliers regarding inventories of medications or other prescribed items currently in stock. In various embodiments, the computer server also provides payment information for the identified patient and transmits the generated prescription and payment information over a computer network to the supplier.

Within FIG. 2, control passes from block 222 to decision diamond 224 to verify that all required information has been confirmed by doctor 100. If not, control passes back to line 208 and block 212 to repeat the process. If all relevant information has been confirmed as accurate, then control passes to block 226. At block 226, prescription engine 110 sends the prescription in electronic format to the selected pharmacy 128; as noted above, this may include patient payment information for reference by the pharmacy. A confirming electronic message is also sent to doctor 100 and/or the office of doctor 100 for entry into the EMR for such patient. In addition, a text message is sent to patient 118 notifying the patient that the prescription has been issued to the selected pharmacy.

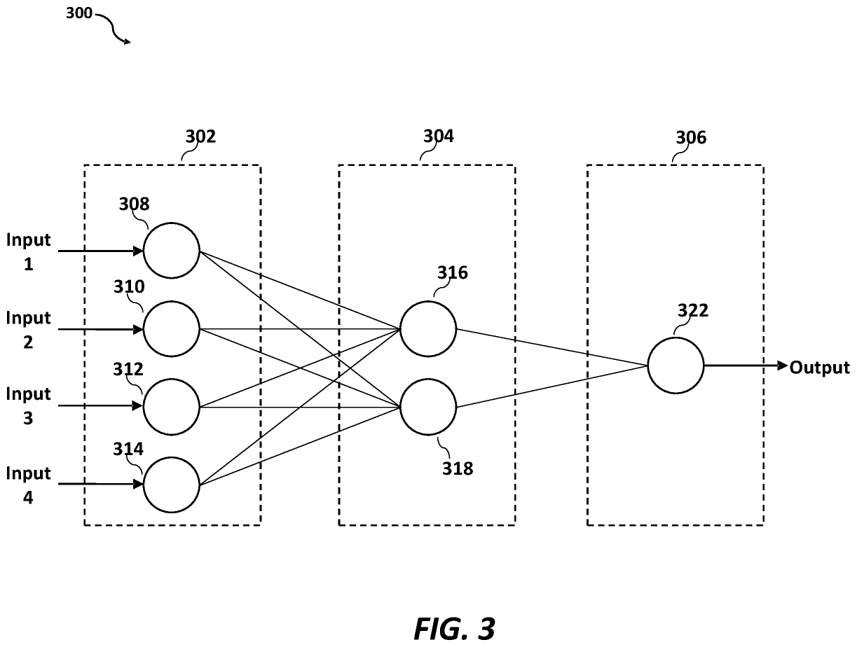

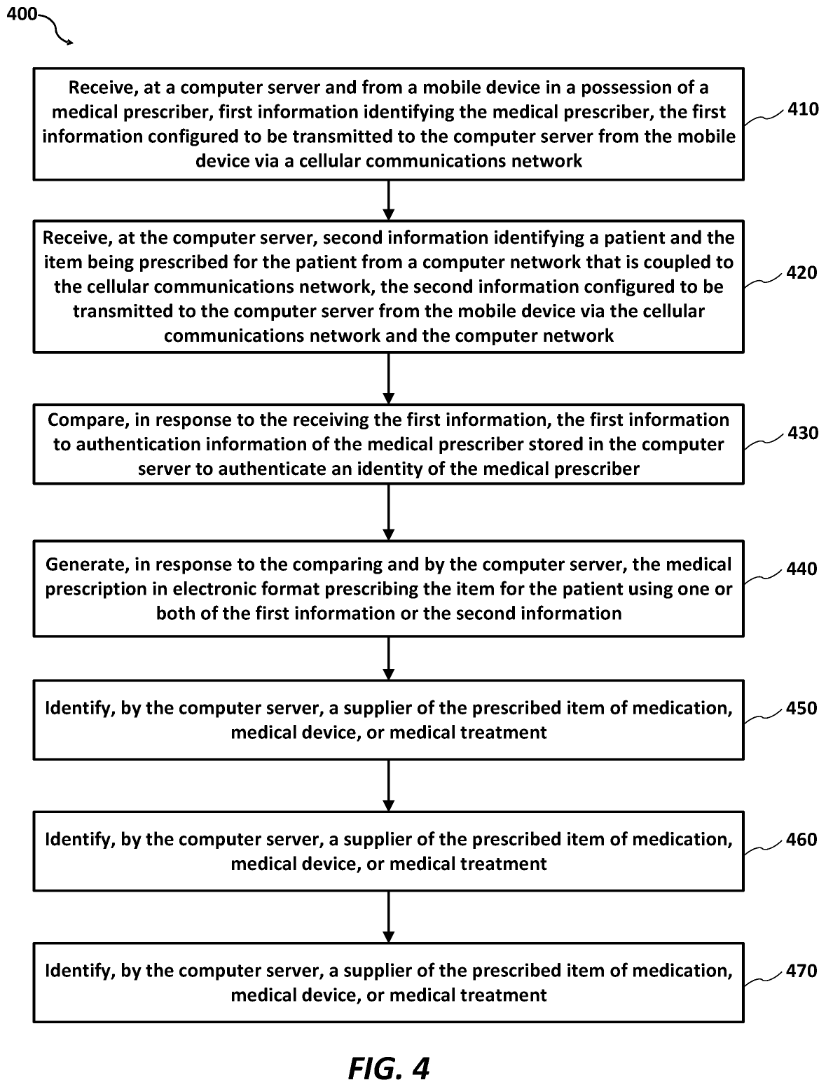

FIG. 3 illustrates an example neural network that can be used to implement a computer-based model according to various embodiments of the present disclosure. In various embodiments, the methods for issuing and filling medical prescriptions for a patient can be implemented via a machine learning/neural network module. That is, as depicted in FIG. 1, the methods (e.g., method 200 in FIG. 2 or method 400 in FIG. 4) disclosed herein can be implemented on a computing device (e.g., automated prescription system 108 or computer server) that includes a prescription engine 110, an authentication engine 112, an NLP engine, an OCR engine, a chatbot engine, a voice recognition engine, a video parsing engine, etc., that may each include a neural network or a machine learning algorithm for executing or implementing the methods for issuing and filling medical prescriptions for a patient discussed herein. FIG. 3 illustrates an example neural network that can be used to implement a computer-based model or engine according to various embodiments of the present disclosure. As shown, the artificial neural network 300 includes three layers—an input layer 302, a hidden layer 304, and an output layer 306. Each of the layers 302, 304, and 306 may include one or more nodes. For example, the input layer 302 includes nodes 308-314, the hidden layer 304 includes nodes 316-318, and the output layer 306 includes a node 322. In this example, each node in a layer is connected to every node in an adjacent layer. For example, the node 308 in the input layer 302 is connected to both of the nodes 316, 318 in the hidden layer 304. Similarly, the node 316 in the hidden layer is connected to all of the nodes 308-314 in the input layer 302 and the node 322 in the output layer 306. Although only one hidden layer is shown for the artificial neural network 300, it has been contemplated that the artificial neural network 300 used to implement the machine learning algorithms of the neural networks included in the prescription engine 110, the authentication engine 112, the NLP engine, the OCR engine, the chatbot engine, the voice recognition engine, the video parsing engine, etc., may include as many hidden layers as necessary or desired.

In this example, the artificial neural network 300 receives a set of input values and produces an output value. Each node in the input layer 302 may correspond to a distinct input value (e.g., different features of the unstructured patient intake data). In some embodiments, each of the nodes 316-318 in the hidden layer 304 generates a representation, which may include a mathematical computation (or algorithm) that produces a value based on the input values received from the nodes 308-314. The mathematical computation may include assigning different weights to each of the data values received from the nodes 308-314. The nodes 316 and 318 may include different algorithms and/or different weights assigned to the data variables from the nodes 308-314 such that each of the nodes 316-318 may produce a different value based on the same input values received from the nodes 308-314. In some embodiments, the weights that are initially assigned to the features (or input values) for each of the nodes 316-318 may be randomly generated (e.g., using a computer randomizer). The values generated by the nodes 316 and 318 may be used by the node 322 in the output layer 306 to produce an output value for the artificial neural network 300. When the artificial neural network 300 is used to implement the machine learning algorithms of the neural networks included in the prescription engine 110, the authentication engine 112, the NLP engine, the OCR engine, the chatbot engine, the voice recognition engine, the video parsing engine, etc., the output value produced by the artificial neural network 300 may include structured patient intake data.

The artificial neural network 300 may be trained by using training data. For example, the training data herein may be unstructured prescription related information or data discussed above (e.g., unstructured text, image, video, audio, etc., that include a prescriber's prescription of medications, medical devices, treatments, etc., for a patient). By providing training data to the artificial neural network 300, the nodes 316-318 in the hidden layer 304 may be trained (adjusted) such that an optimal output is produced in the output layer 306 based on the training data. By continuously providing different sets of training data, and penalizing the artificial neural network 300 when the output of the artificial neural network 300 is incorrect (e.g., when incorrectly identifying or failing to identify unstructured data that can be converted into structured data), the artificial neural network 300 (and specifically, the representations of the nodes in the hidden layer 304) may be trained (adjusted) to improve its performance in data classification. Adjusting the artificial neural network 300 may include adjusting the weights associated with each node in the hidden layer 304.

Although the above discussions pertain to an artificial neural network as an example of machine learning, it is understood that other types of machine learning methods may also be suitable to implement the various aspects of the present disclosure. For example, support vector machines (SVMs) may be used to implement machine learning. SVMs are a set of related supervised learning methods used for classification and regression. A SVM training algorithm—which may be a non-probabilistic binary linear classifier—may build a model that predicts whether a new example falls into one category or another. As another example, Bayesian networks may be used to implement machine learning. A Bayesian network is an acyclic probabilistic graphical model that represents a set of random variables and their conditional independence with a directed acyclic graph (DAG). The Bayesian network could present the probabilistic relationship between one variable and another variable. Another example is a machine learning engine that employs a decision tree learning model to conduct the machine learning process. In some instances, decision tree learning models may include classification tree models, as well as regression tree models. In some embodiments, the machine learning engine employs a Gradient Boosting Machine (GBM) model (e.g., XGBoost) as a regression tree model. Other machine learning techniques may be used to implement the machine learning engine, for example via Random Forest or Deep Neural Networks. Other types of machine learning algorithms are not discussed in detail herein for reasons of simplicity and it is understood that the present disclosure is not limited to a particular type of machine learning.

FIG. 4 is a flowchart illustrating a method 400 for issuing a prescription for a prescriber's patient according to various aspects of the present disclosure. Steps of the method 400 can be executed by a computing device (e.g., a processor, processing circuit, and/or other suitable component) of a computer server or other suitable means for performing the steps. For example, the automated prescription system 108 may utilize one or more components, such as the prescription engine 110, the authentication engine 112, the databases 114, 116, an OCR engine, an NLP engine, etc., which may be part of the automated prescription system 108 or coupled thereto, to execute the steps of method 400. As illustrated, the method 400 includes a number of enumerated steps, but embodiments of the method 400 may include additional steps before, after, and in between the enumerated steps. In some embodiments, one or more of the enumerated steps may be omitted or performed in a different order.

At step 410, the computer server may receive, at a computer server and from a mobile device in a possession of a medical prescriber, first information identifying the medical prescriber, the first information configured to be transmitted to the computer server from the mobile device via a cellular communications network.

At step 420, the computer server may receive, at the computer server, second information identifying a patient and the item being prescribed for the patient from a computer network that is coupled to the cellular communications network, the second information configured to be transmitted to the computer server from the mobile device via the cellular communications network and the computer network.

At step 430, the computer server may compare, in response to the receiving the first information, the first information to authentication information of the medical prescriber stored in the computer server to authenticate an identity of the medical prescriber.

At step 440, the computer server may generate, in response to the comparing and by the computer server, the medical prescription in electronic format prescribing the item for the patient using one or both of the first information or the second information.

At step 450, the computer server may identify, by the computer server, a supplier of the prescribed item of medication, medical device, or medical treatment.

At step 460, the computer server may determine, by the computer server, payment information of the patient.

At step 470, the computer server may transmit the medical prescription and the payment information in electronic format from the computer server to a computer system of the supplier via the computer network.

Some embodiments of the present disclosure disclose a system for allowing a medical prescriber to issue a medical prescription to a supplier of an item of medication, medical device, or medical treatment. The system may comprising in combination: a mobile device for use by the medical prescriber, the mobile device including a cellular transceiver for communicating with a cellular communications network, and the mobile device being adapted to transmit and receive electronic messages over the cellular communications network, the mobile device being configured to permit the medical prescriber to transmit first information identifying the medical prescriber and second information identifying a patient and the item being prescribed for the patient; a computer network coupled to the cellular communications network; and a computer server including a data processor, a transceiver, and data storage, the computer server being coupled with the computer network for exchanging data over the computer network, and for communicating with the mobile device of the medical prescriber. In some embodiments, the computer server further includes: an authentication logic configured to authenticate an identity of the medical prescriber based on a comparison of the first information to authentication information of the medical prescriber stored in the computer server; and a prescription logic configured to generate, based on the comparison, the medical prescription in electronic format prescribing the item for the patient using one or both of the first information or the second information. Further, the computer server may be configured to identify a supplier of the prescribed item of medication, medical device, or medical treatment; determine payment information of the patient; and transmit, via the transceiver, the medical prescription and the payment information in electronic format to a computer system of the supplier via the computer network.

Some embodiments of the present disclosure disclose a non-transitory computer-readable medium (CRM) having stored thereon computer-readable instructions executable to cause performance of operations. In some embodiments, the operations may comprise receiving, at a computer server and from a mobile device in a possession of a medical prescriber, first information identifying the medical prescriber, the first information configured to be transmitted to the computer server from the mobile device via a cellular communications network; receiving, at the computer server, second information identifying a patient and the item being prescribed for the patient from a computer network that is coupled to the cellular communications network, the second information configured to be transmitted to the computer server from the mobile device via the cellular communications network and the computer network; comparing, in response to the receiving the first information, the first information to authentication information of the medical prescriber stored in the computer server to authenticate an identity of the medical prescriber; generating, in response to the comparing and by the computer server, the medical prescription in electronic format prescribing the item for the patient using one or both of the first information or the second information; identifying, by the computer server, a supplier of the prescribed item of medication, medical device, or medical treatment; determining, by the computer server, payment information of the patient; and transmitting the medical prescription and the payment information in electronic format from the computer server to a computer system of the supplier via the computer network.

In some embodiments of method 400 or the afore-mentioned operations, the identifying the supplier includes identifying one or more of the supplier that is in closest proximity to the patient, the supplier qualified to fill the medical prescription, the supplier that has the prescribed item in stock, or the supplier that has the prescribed item at the lowest price.

In some embodiments, the second information is included in an image file configured to be transmitted to the computer server from the mobile device via the cellular communications network and the computer network. In such cases, the method 400 or the operations may further comprise: extracting the second information from the image using an optical character recognition (OCR) engine of the computer server.

In some embodiments, the second information is included in a text message configured to be transmitted to the computer server from the mobile device via the cellular communications network and the computer network. In such cases, the method 400 or the operations may further comprise extracting the second information from the image using a natural language processing (NLP) engine of the computer server.

In some embodiments, the second information is included in an audio file configured to be transmitted to the computer server from the mobile device via the cellular communications network and the computer network. In such cases, the method 400 or the operations may further comprise extracting the second information from the audio file using an artificial intelligence voice recognition engine of the computer server.

In some embodiments, the generating the medical prescription in electronic format includes populating fields of an electronic prescription form at the computer server with the one or both of the first information or the second information.

In some embodiments, the second information is configured to be transmitted to the computer server from the mobile device via the cellular communications network and the computer network via an encrypted bi-directional communication link configured to anonymize data communicated therewithin.

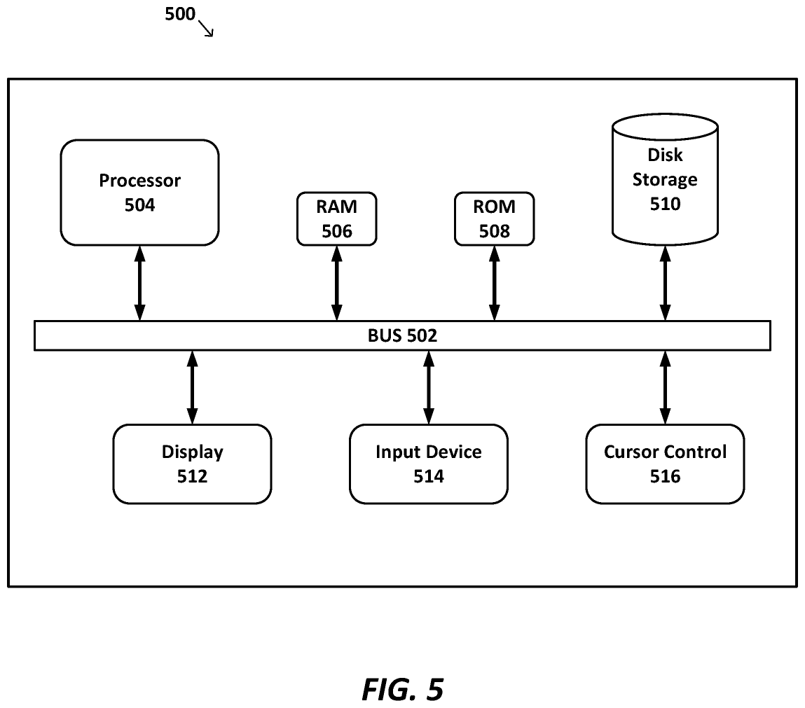

In some embodiments, the methods for issuing and filling medical prescriptions for a patient can be implemented via computer software or hardware. In various embodiments, the methods for issuing and filling medical prescriptions for a patient can be implemented via a machine learning/neural network module. That is, as depicted in FIG. 1, the methods (e.g., method 200 in FIG. 2 or method 400 in FIG. 4) disclosed herein can be implemented on a computing device (e.g., automated prescription system 108 or computer server) that includes a prescription engine 110, an authentication engine 112, an NLP engine, an OCR engine, a chatbot engine, a voice recognition engine, a video parsing engine, etc. In some embodiments, the computing devices shown in FIG. 1, such as the mobile device 102, the mobile device 120, the computer device or system of the human live support 130, the prescription engine 110, the authentication engine 112, the databases or storage systems 114, 116, the cell tower 104, 122, etc., may at least in part be implemented via the example computer system 500 shown in FIG. 5.

It should be appreciated that the various engines depicted in FIG. 1 can be combined or collapsed into a single engine, component or module, depending on the requirements of the particular application or system architecture. Moreover, in various embodiments, the memory, a prescription engine 110, an authentication engine 112, an NLP engine, an OCR engine, a chatbot engine, a voice recognition engine, a video parsing engine, etc., can comprise additional engines or components as needed by the particular application or system architecture.

FIG. 5 is a block diagram illustrating a computer system 500 upon which embodiments of the present teachings may be implemented. In various embodiments of the present teachings, computer system 500 can include a bus 502 or other communication mechanism for communicating information and a processor 504 coupled with bus 502 for processing information. In various embodiments, computer system 500 can also include a memory, which can be a random-access memory (RAM) 506 or other dynamic storage device, coupled to bus 502 for determining instructions to be executed by processor 504. Memory can also be used for storing temporary variables or other intermediate information during execution of instructions to be executed by processor 504. In various embodiments, computer system 500 can further include a read only memory (ROM) 508 or other static storage device coupled to bus 502 for storing static information and instructions for processor 504. A storage device 510, such as a magnetic disk or optical disk, can be provided and coupled to bus 502 for storing information and instructions.

In various embodiments, computer system 500 can be coupled via bus 502 to a display 512, such as a cathode ray tube (CRT) or liquid crystal display (LCD), for displaying information to a computer user. An input device 514, including alphanumeric and other keys, can be coupled to bus 502 for communication of information and command selections to processor 504. Another type of user input device is a cursor control 516, such as a mouse, a trackball or cursor direction keys for communicating direction information and command selections to processor 504 and for controlling cursor movement on display 512. This input device 514 typically has two degrees of freedom in two axes, a first axis (i.e., x) and a second axis (i.e., y), that allows the device to specify positions in a plane. However, it should be understood that input devices 514 allowing for 3-dimensional (x, y and z) cursor movement are also contemplated herein.

Consistent with certain implementations of the present teachings, results can be provided by computer system 500 in response to processor 504 executing one or more sequences of one or more instructions contained in memory 506. Such instructions can be read into memory 506 from another computer-readable medium or computer-readable storage medium, such as storage device 510. Execution of the sequences of instructions contained in memory 506 can cause processor 504 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 504 for execution. Such a medium can take many forms, including but not limited to, non-volatile media, volatile media, and transmission media. Examples of non-volatile media can include, but are not limited to, dynamic memory, such as memory 506. Examples of transmission media can include, but are not limited to, coaxial cables, copper wire, and fiber optics, including the wires that comprise bus 502.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, another memory chip or cartridge, or any other tangible medium from which a computer can read.

In addition to computer-readable medium, instructions or data can be provided as signals on transmission media included in a communications apparatus or system to provide sequences of one or more instructions to processor 504 of computer system 500 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein, flow charts, diagrams and accompanying disclosure can be implemented using computer system 500 as a standalone device or on a distributed network or shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 500, whereby processor 504 would execute the analyses and determinations provided by these engines, subject to instructions provided by any one of, or a combination of, memory components 506/508/510 and user input provided via input device 514.

### RECITATIONS OF SOME EMBODIMENTS OF THE PRESENT DISCLOSURE

Embodiment 1: A method for allowing a medical prescriber to issue a medical prescription to a supplier of an item of medication, medical device, or medical treatment, the method comprising: receiving, at a computer server and from a mobile device in a possession of a medical prescriber, first information identifying the medical prescriber, the first information configured to be transmitted to the computer server from the mobile device via a cellular communications network;

receiving, at the computer server, second information identifying a patient and the item being prescribed for the patient from a computer network that is coupled to the cellular communications network, the second information configured to be transmitted to the computer server from the mobile device via the cellular communications network and the computer network; comparing, in response to the receiving the first information, the first information to authentication information of the medical prescriber stored in the computer server to authenticate an identity of the medical prescriber; generating, in response to the comparing and by the computer server, the medical prescription in electronic format prescribing the item for the patient using one or both of the first information or the second information; identifying, by the computer server, a supplier of the prescribed item of medication, medical device, or medical treatment; determining, by the computer server, payment information of the patient; and transmitting the medical prescription and the payment information in electronic format from the computer server to a computer system of the supplier via the computer network.

Embodiment 2: The method of embodiment 1, wherein the identifying the supplier includes identifying one or more of the supplier that is in closest proximity to the patient, the supplier qualified to fill the medical prescription, the supplier that has the prescribed item in stock, or the supplier that has the prescribed item at the lowest price.

Embodiment 3: The method of embodiment 1 or 2, wherein the second information is included in an image file configured to be transmitted to the computer server from the mobile device via the cellular communications network and the computer network, the method further comprising: extracting the second information from the image using an optical character recognition (OCR) engine of the computer server.

Embodiment 4: The method of any of embodiments 1-3, wherein the second information is included in a text message configured to be transmitted to the computer server from the mobile device via the cellular communications network and the computer network, the method further comprising: extracting the second information from the image using a natural language processing (NLP) engine of the computer server.

Embodiment 5: The method of any of embodiments 1-4, wherein the second information is included in an audio file configured to be transmitted to the computer server from the mobile device via the cellular communications network and the computer network, the method further comprising: extracting the second information from the audio file using an artificial intelligence voice recognition engine of the computer server.

Embodiment 6: The method of any of embodiments 1-5, wherein the generating the medical prescription in electronic format includes populating fields of an electronic prescription form at the computer server with the one or both of the first information or the second information.

Embodiment 7: The method of any of embodiments 1-6, wherein the second information is configured to be transmitted to the computer server from the mobile device via the cellular communications network and the computer network via an encrypted bi-directional communication link configured to anonymize data communicated therewithin.

Embodiment 9: A system, comprising: a mobile device, a computer network, and a computer server, configured to perform the methods of embodiments 1-8.

Embodiment 10: A non-transitory machine-readable medium having stored thereon machine-readable instructions executable to cause a machine to perform the methods of embodiments 1-8.

It will be recognized that an improved system and method have now been described for issuing and filling medical prescriptions for a patient using one or more mobile devices. The embodiments specifically illustrated and/or described herein are provided merely to exemplify particular applications of the invention. These descriptions and drawings should not be considered in a limiting sense, as it is understood that the present invention is in no way limited to only the disclosed embodiments. While some examples herein are directed to eye care physicians, those skilled in the art will appreciate that the invention may be applied to virtually any field of health care which involves the prescription of medications, medical devices, or medical treatment. While examples have been described wherein the prescription relates to medication and the supplier is a pharmacy, it will be appreciated that the prescription might also relate to a medical device (e.g., a wheelchair, eyeglasses, contact lenses, orthotics, hearing aids, therapeutic devices, etc.), and the supplier could be a laboratory or a medical supply house. Also, the prescription could relate to medical treatment or medical diagnostic tests (e.g., a referral to a specialist, physical therapy, X-ray, CT scan, MRI, dialysis, etc.).

Similarly, while examples described herein relate to patients who are human beings, those skilled in the art will appreciate that the invention may be applied in the veterinary field to medications and/or medical devices prescribed for animals. It will be appreciated that other modifications or adaptations of the methods and or specific structures described herein may become apparent to those skilled in the art. All such modifications, adaptations, or variations are considered to be within the spirit and scope of the present invention, and within the scope of the appended claims.

What is claimed is:

1. A system for allowing a medical prescriber to issue a medical prescription to a supplier of an item of medication, medical device, or medical treatment, comprising in combination:
a mobile device for use by the medical prescriber, the mobile device including a cellular transceiver for communicating with a cellular communications network, and the mobile device being adapted to transmit and receive electronic messages over the cellular communications network, the mobile device being configured to permit the medical prescriber to transmit first information identifying the medical prescriber and second information identifying a patient and the item being prescribed for the patient;
a computer network coupled to the cellular communications network; and
a computer server including a data processor, a transceiver, and data storage, the computer server being coupled with the computer network for exchanging data over the computer network, and for communicating with the mobile device of the medical prescriber,
the computer server further including:
an authentication logic configured to authenticate an identity of the medical prescriber based on a comparison of the first information to authentication information of the medical prescriber stored in the computer server; and
a prescription logic configured to generate, based on the comparison, the medical prescription in electronic format prescribing the item for the patient using one or both of the first information or the second information; and
the computer server further configured to:
identify a supplier of the prescribed item of medication, medical device, or medical treatment;
determine payment information of the patient; and
transmit, via the transceiver, the medical prescription and the payment information in electronic format to a computer system of the supplier via the computer network.

2. The system of claim 1, wherein the computer server is further configured to identify one or more of the supplier that is in closest proximity to the patient, the supplier qualified to fill the medical prescription, the supplier that has the prescribed item in stock, or the supplier that has the prescribed item at the lowest price.

3. The system of claim 1, wherein the second information is included in an image file configured to be transmitted to the computer server from the mobile device via the cellular communications network and the computer network, the computer server further comprising:
an optical character recognition (OCR) engine configured to extract the second information from the image.

4. The system of claim 1, wherein the second information is included in a text message configured to be transmitted to the computer server from the mobile device via the cellular communications network and the computer network, the computer server further comprising:
a natural language processing (NLP) engine configured to extract the second information from the text message.

5. The system of claim 1, wherein the second information is included in an audio file configured to be transmitted to the computer server from the mobile device via the cellular communications network and the computer network, the computer server further comprising:
an artificial intelligence voice recognition engine configured to extract the second information from the audio file.

6. The system of claim 1, wherein the computer server is further configured to populate fields of an electronic prescription form at the computer server with the one or both of the first information or the second information.

7. The system of claim 1, wherein the mobile device is configured to permit the medical prescriber to transmit the second information to the computer server via an encrypted bi-directional communication link configured to anonymize data communicated therewithin.

8. A method for allowing a medical prescriber to issue a medical prescription to a supplier of an item of medication, medical device, or medical treatment, the method comprising:
receiving, at a computer server and from a mobile device in a possession of a medical prescriber, first information identifying the medical prescriber, the first information configured to be transmitted to the computer server from the mobile device via a cellular communications network;
receiving, at the computer server, second information identifying a patient and the item being prescribed for the patient from a computer network that is coupled to the cellular communications network, the second information configured to be transmitted to the computer server from the mobile device via the cellular communications network and the computer network;
comparing, in response to the receiving the first information, the first information to authentication information of the medical prescriber stored in the computer server to authenticate an identity of the medical prescriber;
generating, in response to the comparing and by the computer server, the medical prescription in electronic format prescribing the item for the patient using one or both of the first information or the second information;
identifying, by the computer server, a supplier of the prescribed item of medication, medical device, or medical treatment;
determining, by the computer server, payment information of the patient; and
transmitting the medical prescription and the payment information in electronic format from the computer server to a computer system of the supplier via the computer network.

9. The method of claim 8, wherein the identifying the supplier includes identifying one or more of the supplier that is in closest proximity to the patient, the supplier qualified to fill the medical prescription, the supplier that has the prescribed item in stock, or the supplier that has the prescribed item at the lowest price.

10. The method of claim 8, wherein the second information is included in an image file configured to be transmitted to the computer server from the mobile device via the cellular communications network and the computer network, the method further comprising:
extracting the second information from the image using an optical character recognition (OCR) engine of the computer server.

11. The method of claim 8, wherein the second information is included in a text message configured to be transmitted to the computer server from the mobile device via the cellular communications network and the computer network, the method further comprising:
extracting the second information from the image using a natural language processing (NLP) engine of the computer server.

12. The method of claim 8, wherein the second information is included in an audio file configured to be transmitted to the computer server from the mobile device via the cellular communications network and the computer network, the method further comprising:
extracting the second information from the audio file using an artificial intelligence voice recognition engine of the computer server.

13. The method of claim 8, wherein the generating the medical prescription in electronic format includes populating fields of an electronic prescription form at the computer server with the one or both of the first information or the second information.

14. The method of claim 8, wherein the second information is configured to be transmitted to the computer server from the mobile device via the cellular communications network and the computer network via an encrypted bi-directional communication link configured to anonymize data communicated therewithin.

15. A non-transitory computer-readable medium (CRM) having stored thereon computer-readable instructions executable to cause performance of operations comprising:
receiving, at a computer server and from a mobile device in a possession of a medical prescriber, first information identifying the medical prescriber, the first information configured to be transmitted to the computer server from the mobile device via a cellular communications network;
receiving, at the computer server, second information identifying a patient and the item being prescribed for the patient from a computer network that is coupled to the cellular communications network, the second information configured to be transmitted to the computer server from the mobile device via the cellular communications network and the computer network;
comparing, in response to the receiving the first information, the first information to authentication information of the medical prescriber stored in the computer server to authenticate an identity of the medical prescriber;
generating, in response to the comparing and by the computer server, the medical prescription in electronic format prescribing the item for the patient using one or both of the first information or the second information;
identifying, by the computer server, a supplier of the prescribed item of medication, medical device, or medical treatment;
determining, by the computer server, payment information of the patient; and
transmitting the medical prescription and the payment information in electronic format from the computer server to a computer system of the supplier via the computer network.

16. The non-transitory CRM of claim 15, wherein the identifying the supplier includes identifying one or more of the supplier that is in closest proximity to the patient, the supplier qualified to fill the medical prescription, the supplier that has the prescribed item in stock, or the supplier that has the prescribed item at the lowest price.

17. The met non-transitory CRM of claim 15, wherein the second information is included in an image file configured to be transmitted to the computer server from the mobile device via the cellular communications network and the computer network, the method further comprising:
extracting the second information from the image using an optical character recognition (OCR) engine of the computer server.

18. The non-transitory CRM of claim 15, wherein the second information is included in a text message configured to be transmitted to the computer server from the mobile device via the cellular communications network and the computer network, the method further comprising:
extracting the second information from the image using a natural language processing (NLP) engine of the computer server

19. The non-transitory CRM of claim 15, wherein the second information is included in an audio file configured to be transmitted to the computer server from the mobile device via the cellular communications network and the computer network, the method further comprising:
extracting the second information from the audio file using an artificial intelligence voice recognition engine of the computer server.

20. The non-transitory CRM of claim 15, wherein the generating the medical prescription in electronic format includes populating fields of an electronic prescription form at the computer server with the one or both of the first information or the second information.
